(19) United States
(12) Patent Application Publication
(10) Pub. No.: US 2023/0031188 A1
(43) Pub. Date: Feb. 2, 2023

(54) Methods of Treating Skin Diseases

(71) Applicant: Adam Benkirane, Santa Clara, CA (US)

(72) Inventor: Adam Benkirane, Santa Clara, CA (US)

(21) Appl. No.: 17/869,482

(22) Filed: Jul. 20, 2022

Related U.S. Application Data
(60) Provisional application No. 63/203,741, filed on Jul. 29, 2021.

(51) Int. Cl. B01D 11/02; A61K 47/44; A61K 47/26; A61K 9/06; A61K 9/00; A61P 17/00; A61K 36/77

(52) U.S. Cl. CPC B01D 11/0288; A61K 47/44; A61K 47/26; A61K 9/06; A61K 9/0014; A61P 17/00; A61K 36/77

(57) ABSTRACT

Methods of treating or preventing skin diseases, such as for example, lichen sclerosus, pruritis or dysuria in a patient are provided. A therapeutically effective amount of a Peganum harmala extract or a pharmaceutical composition thereof is administered to the patient in need thereof. Also provided are methods for making a Peganum harmala extract and pharmaceutical compositions thereof.

### CROSS-REFERENCE TO RELATED APPLICATION(S)

This application claims priority to and the benefit of U.S. Provisional Application Patent Ser. No. 63/203,741, filed Jul. 29, 2021, the entire disclosure of which is hereby incorporated by reference.

### FIELD

Methods of treating or preventing skin diseases, such as for example, lichen sclerosus, pruritis or dysuria in a patient are provided. A therapeutically effective amount of a Peganum harmala extract or a pharmaceutical composition thereof is administered to the patient in need thereof. Also provided are methods for making a Peganum harmala extract and pharmaceutical compositions thereof.

### BACKGROUND

Lichen sclerosus is a rare skin disease which often affects the genitals and can lead to an increased risk of genital cancers if left untreated. As of this time, the underlying etiology of the disease is not defined. Some practitioners believe lichen sclerosus may be caused by an autoimmune disorder. Contrastingly, some evidence suggests that lichen sclerosus is associated with Lyme disease.

Some symptoms associated with lichen sclerosus include thinning of the skin, increased susceptibility to tearing of affected skin, depigmentation, pruritis, dysuria, sexual dysfunction, contraction/constriction of skin, fusing of contracted skin and/or plaque-like growths on the skin.

Current treatment options can remedy some symptoms, but have significant issues. Patients suffering from lichen sclerosus are typically treated with topical steroids. Use of topical steroids reduces the probability of genital cancer in patients suffering from lichen sclerosus and often relieves many of the symptoms associated with lichen sclerosus. However, administration of steroids fails to treat all lichen sclerosus symptoms and in some situations does not alleviate any of the symptoms associated with lichen sclerosus.

Furthermore, steroid administration can lead to significant side effects including, for example, thinning of the skin, masking of candidiasis symptoms, decrease libido, skin irritation, etc. Accordingly, what is needed are new methods for treating lichen sclerosus, which successfully treat the disease without having significant side effects.

### SUMMARY

The disclosure provided herein satisfies these and other needs. In one aspect, a Peganum harmala extract is provided. The extract is made by a process which includes the steps of crushing dry seeds of Peganum harmala, adding an organic solvent to the crushed seeds to form a suspension, letting the suspension stand until the suspension is a dark red color, filtering the suspension and removing the solvent. In some embodiments, the suspension is agitated.

In another aspect, a pharmaceutical composition is provided. The pharmaceutical composition includes a Peganum harmala extract and a pharmaceutically acceptable vehicle.

In still another aspect, a method of treating lichen sclerosus is provided. A therapeutically effective amount of a Peganum harmala extract or a therapeutically effective amount of a pharmaceutical composition which includes a Peganum harmala extract is administered to a patient in need thereof.

In still another aspect, a method of treating pruritis is provided. A therapeutically effective amount of a Peganum harmala extract or a therapeutically effective amount of a pharmaceutical composition which includes a Peganum harmala extract is administered to a patient in need thereof. In some embodiments, the patient is suffering from lichen sclerosus.

In still another aspect, a method of treating dysuria is provided. A therapeutically effective amount of a Peganum harmala extract or a therapeutically effective amount of a pharmaceutical composition which includes a Peganum harmala extract is administered to a patient in need thereof. In some embodiments, the patient is suffering from lichen sclerosus.

In still another aspect, methods of reducing tearing of affected skin, and treating thinning of skin, depigmentation, sexual dysfunction, contraction/constriction of skin, fusing of contracted skin or plaque like growths on the skin are provided. A therapeutically effective amount of a Peganum harmala extract or a therapeutically effective amount of a pharmaceutical composition which includes a Peganum harmala extract is administered to a patient in need thereof. In some embodiments, the patient is suffering from lichen sclerosus.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values.

“Preventing,” or “prevention,” refers to a reduction in risk of acquiring a disease or disorder (i.e., causing at least one of the clinical symptoms of the disease not to develop in a patient that may be exposed to or predisposed to the disease but does not yet experience or display symptoms of the disease). The application of a therapeutic for preventing or prevention of a disease or disorder is known as ‘prophylaxis.’ In some embodiments, the Peganum harmala extracts disclosed herein provide superior prophylaxis because of fewer long term side effects.

“Subject,” “individual,” or “patient,” is used interchangeably herein and refers to a vertebrate, preferably a mammal. Mammals include, but are not limited to, murines, rodents, simians, humans, farm animals, sport animals and pets.

“Treating,” or “treatment,” of any disease or disorder refers, in some embodiments, to ameliorating the disease or disorder (i.e., arresting or reducing the development of the disease or at least one of the clinical symptoms thereof. Treatment may also be considered to include preemptive or prophylactic administration to ameliorate, arrest or prevent the development of the disease or at least one of the clinical symptoms. In a further feature, the treatment rendered has lower potential for long-term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount,” means the amount of Peganum harmala extract that, when administered to a patient for treating a disease, is sufficient to treat the disease. The “therapeutically effective amount” will vary depending on the disease and its severity and the age, weight, adsorption, distribution, metabolism and excretion etc., of the patient to be treated.

“Vehicle,” refers to a diluent, excipient or carrier with which the Peganum harmala extract is administered to a subject. In some embodiments, the vehicle is pharmaceutically acceptable.

### Methods of Use

Peganum harmala extracts have been used in traditional healing. However, treating lichen sclerosus with Peganum harmala extracts is entirely without precedence.

In some embodiments, a Peganum harmala extract made by a process comprising the steps of crushing dry seeds of Peganum harmala, adding an organic solvent to the crushed seeds to form a suspension, letting the suspension stand until it is a dark red color, filtering the suspension and removing the solvent is provided. In some embodiments, the suspension is agitated on at least a daily basis.

In some embodiments, the solvent is ethanol. However, a number of different solvents and/or solvent combinations may be used to form Peganum harmala extracts. The most important consideration is adequately extracting the various chemical components from the seeds of the Peganum harmala plant which is well within the ambit of those of skill in the art.

In some embodiments, a pharmaceutical composition is provided. The pharmaceutical composition includes a Peganum harmala extract and a pharmaceutically acceptable vehicle. In some embodiments, the pharmaceutically acceptable vehicle is unrefined coconut oil.

In some embodiments, the ratio of Peganum harmala extract to the pharmaceutically effective vehicle is about 1 to about 50. In other embodiments, the ratio of Peganum harmala extract to the pharmaceutically effective vehicle is about 1 to about 20. In still other embodiments, the ratio of Peganum harmala extract to the pharmaceutically effective vehicle is about 1 to about 12. In still other embodiments, the ratio of Peganum harmala extract to the pharmaceutically effective vehicle is about 1 to about 10. In still other embodiments, the ratio of Peganum harmala extract to the pharmaceutically effective vehicle is about 1 to about 3. In still other embodiments, the ratio of Peganum harmala extract to the pharmaceutically effective vehicle is about 1 to about 2.

### Compositions and Methods of Administration

The compositions provided herein contain therapeutically effective amounts of the Peganum harmala extract provided herein that are useful in the prevention, treatment, or amelioration of one or more of the symptoms of diseases or disorders described herein and a vehicle. Vehicles suitable for administration of the extract provided herein include any such carriers known to those skilled in the art to be suitable for the particular mode of administration. In addition, the extract may be formulated as the sole active ingredient in the composition or may be combined with other active ingredients such as for example, clove oil.

The Peganum harmala extract is, in some embodiments, formulated into suitable preparations such as solutions, suspensions, tablets, dispersible tablets, pills, capsules, powders, sustained release formulations or elixirs, for oral administration or in sterile solutions or suspensions for parenteral administration, as well as topical administration, transdermal administration and oral inhalation via nebulizers, pressurized metered dose inhalers and dry powder inhalers. In some embodiments, the Peganum harmala extract described is formulated into compositions using techniques and procedures well known in the art (see, e.g., Ansel, Introduction to Pharmaceutical Dosage Forms, Seventh Edition (1999)).

In the compositions, an effective concentration of the Peganum harmala extract is mixed with a suitable vehicle. The concentration of the Peganum harmala extract in the compositions is effective for delivery of an amount, upon administration that treats, leads to prevention, or amelioration of one or more of the symptoms of diseases or disorders described herein. In some embodiments, the compositions are formulated for single dosage administration. To formulate a composition, the weight fraction of the Peganum harmala extract is dissolved, suspended, dispersed or otherwise mixed in a selected vehicle at an effective concentration such that the treated condition is relieved, prevented, or one or more symptoms are ameliorated.

The Peganum harmala extract is included in the vehicle in an amount sufficient to exert a therapeutically useful effect in the absence of undesirable side effects on the patient being treated. The therapeutically effective concentration may be predicted empirically by testing the Peganum harmala extract in in vitro and in vivo systems well known to those of skill in the art and then extrapolated therefrom for dosages for humans. Human doses are then typically finetuned in clinical trials and titrated to response.

The concentration of the Peganum harmala extract in the composition will depend on absorption, inactivation and excretion rates of the active components of the extract, the physicochemical characteristics of the Peganum harmala extract, the dosage schedule, and amount administered as well as other factors known to those of skill in the art. For example, the amount that is delivered is sufficient to ameliorate one or more of the symptoms of diseases or disorders as described herein.

In instances in which the Peganum harmala extract exhibits insufficient solubility, methods for solubilizing may be used such as use of nanoparticles, or emulsions or tertiary templating. Such methods are known to those of skill in this art, and include, but are not limited to, using cosolvents, such as dimethylsulfoxide (DMSO), clove oil, using surfactants or surface modifiers, such as TWEEN®, complexing agents such as cyclodextrin or dissolution by enhanced ionization (i.e., dissolving in aqueous sodium bicarbonate).

Upon mixing or addition of the Peganum harmala extract the resulting mixture may be a solution, suspension, emulsion or the like. The form of the resulting mixture depends upon a number of factors, including the intended mode of administration and the solubility of the Peganum harmala extract in the selected vehicle. The effective concentration is sufficient for ameliorating the symptoms of the disease, disorder or condition treated and may be empirically determined.

The compositions are provided for administration to humans and animals in indication appropriate dosage forms, such as dry powder inhalers (DPIs), pressurized metered dose inhalers (pMDIs), nebulizers, tablets, capsules, pills, sublingual tapes/bioerodible strips, tablets or capsules, powders, granules, lozenges, lotions, salves, suppositories, fast melts, transdermal patches or other transdermal application devices/preparations, sterile parenteral solutions or suspensions, and oral solutions or suspensions, and oil water emulsions containing suitable quantities of the Peganum harmala extract. The Peganum harmala extracts are, in some embodiments, formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active Peganum harmala extract sufficient to produce the desired therapeutic effect, in association with the required vehicle. Examples of unit dose forms include ampoules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Liquid compositions can, for example, be prepared by dissolving, dispersing, or otherwise mixing the Peganum harmala extract and optional adjuvants in a vehicle, such as, for example, water, saline, aqueous dextrose, glycerol, glycols, ethanol, and the like, to thereby form a solution or suspension, colloidal dispersion, emulsion or liposomal formulation. If desired, the composition to be administered may also contain minor amounts of nontoxic auxiliary substances such as wetting agents, emulsifying agents, solubilizing agents, pH buffering agents and the like, for example, acetate, sodium citrate, cyclodextrin derivatives, DMSO, clove oil, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 100% with the balance made up from vehicle or carrier may be prepared. Methods for preparation of these compositions are known to those skilled in the art. The contemplated compositions may contain 0.001% to 100% active ingredient.

In certain embodiments, the compositions are lactose free compositions containing excipients that are well known in the art and are listed, for example, in the U.S. Pharmacopeia (USP) 25NF20 (2002). In general, lactose free compositions contain active ingredients, a binder/filler, and a lubricant in compatible amounts. Particular lactose free dosage forms contain active ingredients, microcrystalline cellulose, pregelatinized starch, and magnesium stearate.

Oral dosage forms are either solid, gel or liquid. The solid dosage forms are tablets, capsules, granules, and bulk powders. Types of oral tablets include compressed, chewable lozenges and tablets which may be enteric coated, sugarcoated or filmcoated. Capsules may be hard or soft gelatin capsules, while granules and powders may be provided in no effervescent or effervescent form with the combination of other ingredients known to those skilled in the art.

In certain embodiments, the formulations are solid dosage forms such as for example, capsules or tablets. The tablets, pills, capsules, troches and the like can contain Peganum harmala extract: a binder; a lubricant; a diluent; a glidant; a disintegrating agent; a coloring agent; a sweetening agent; a flavoring agent; a wetting agent; an enteric coating; a film coating agent and modified release agent. Examples of binders include microcrystalline cellulose, methyl paraben, polyalkyleneoxides, gum tragacanth, glucose solution, acacia mucilage, gelatin solution, molasses, polyvinylpyrrolidine, povidone, crospovidones, sucrose and starch and starch derivatives. Lubricants include talc, starch, magnesium/calcium stearate, lycopodium and stearic acid. Diluents include, for example, lactose, sucrose, trehalose, lysine, leucine, lecithin, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate and advanced coloring or anti- forgery color/opalescent additives known to those skilled in the art. Sweetening agents include sucrose, lactose, mannitol and artificial sweetening agents such as saccharin and any number of spray dried flavors. Flavoring agents include natural flavors extracted from plants such as fruits and synthetic blends of compounds which produce a pleasant sensation or mask unpleasant taste, such as, but not limited to peppermint and methyl salicylate. Wetting agents include propylene glycol monostearate, sorbitan monooleate, diethylene glycol monolaurate and polyoxyethylene lauryl ether. Enteric coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate. Modified release agents include polymers such as the Eudragit® series and cellulose esters.

The Peganum harmala extract can be provided in a composition that protects it from the acidic environment of the stomach. For example, the composition can be formulated in an enteric coating that maintains its integrity in the stomach and releases the Peganum harmala extract in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The Peganum harmala extract can also be administered as a component of an elixir, suspension, syrup, wafer, sprinkle, chewing gum or the like. A syrup may contain, in addition to the Peganum harmala extract, sucrose as a sweetening agent and certain preservatives, dyes and colorings and flavors.

The active materials can also be mixed with other active materials which do not impair the desired action, or with materials that supplement the desired action, such as antacids, H₂ blockers, and diuretics. Higher concentrations, up to about 98% by weight of the Peganum harmala extract may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from no effervescent granules and effervescent preparations reconstituted from effervescent granules. Aqueous solutions include, for example, elixirs and syrups. Emulsions are either oil in water or water in oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two phase system in which one liquid is dispersed in the form of small globules throughout another liquid. Carriers used in emulsions are nonaqueous liquids, emulsifying agents and preservatives. Suspensions use suspending agents and preservatives. Acceptable substances used in non-effervescent granules, to be reconstituted into a liquid oral dosage form, include diluents, sweeteners and wetting agents. Acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of nonaqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a liquid vehicle, e.g., water, to be easily measured for administration.

Alternatively, liquid or semisolid oral formulations may be prepared by dissolving or dispersing the Peganum harmala extract in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing Peganum harmala extract, a dialkylated mono or polyalkylene glycol, including, but not limited to, 1,2dimethoxyethane, diglyme, triglyme, tetraglyme, polyethylene glycol350dimethyl ether, polyethylene glycol550dimethyl ether, polyethylene glycol750dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of nontoxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow release or sustained release system, such that a constant level of dosage is maintained (see, e.g., U.S. Pat. No. 3,710,795) is also contemplated herein. Briefly, the Peganum harmala extract provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylenevinylacetate copolymers, silicone rubbers, polydimethylsiloxanes, silicone carbonate copolymers, hydrophilic polymers such as hydrogels of esters of acrylic and methacrylic acid, collagen, crosslinked polyvinylalcohol and crosslinked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The Peganum harmala extract diffuses through the outer polymeric membrane in a release rate controlling step.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (Tween® 80). A sequestering or chelating agent of metal ions includes EDTA. Carriers also include ethyl alcohol, polyethylene glycol and propylene glycol for water miscible vehicles; and sodium hydroxide, hydrochloric acid, citric acid or lactic acid for pH adjustment.

The concentration of Peganum harmala extract is adjusted so that an injection provides an effective amount to produce the desired pharmacological effect. The exact dose depends on the age, weight, body surface area and condition of the patient or animal as is known in the art.

The unit dose parenteral preparations are packaged in an ampoule, a vial or a syringe with a needle. All preparations for parenteral administration must be sterile, as is known and practiced in the art.

The Peganum harmala extract may be suspended in micronized or other suitable form or may be derivatized to produce a more soluble active product or to produce a prodrug. The form of the resulting mixture depends upon a number of factors, including the intended mode of administration and the solubility of the Peganum harmala extract in the selected carrier or vehicle. The effective concentration is sufficient for ameliorating the symptoms of the condition and may be empirically determined.

Active ingredients provided herein can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 and 6,740,634. Such dosage forms can be used to provide slow or controlled release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein.

All controlled release products have a common goal of improving drug therapy over that achieved by their noncontrolled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased patient compliance. In addition, controlled release formulations can be used to affect the time of onset of action or other characteristics, such as blood levels of the drug, and can thus affect the occurrence of side (e.g., adverse) effects.

Most controlled release formulations are designed to initially release an active ingredient that promptly produces the desired therapeutic effect, and then gradually continually releases other amounts of Peganum harmala extract to maintain this level of therapeutic or prophylactic effect over an extended period of time. In order to maintain a constant level of Peganum harmala extract in the body, the Peganum harmala extract must be released from the dosage form at a rate that will replace the amount of Peganum harmala extract being metabolized and excreted from the body.

Of interest herein are also lyophilized powders, which can be reconstituted for administration as solutions, emulsions and other mixtures. They may also be reconstituted and formulated as solids or gels.

The sterile, lyophilized powder is prepared by dissolving Peganum harmala extract provided herein in a suitable solvent. The solvent may contain an excipient which improves the stability or other pharmacological components of the powder or reconstituted solution, prepared from the powder. Excipients that may be used include, but are not limited to, an antioxidant, a buffer and a bulking agent. In some embodiments, the excipient is selected from dextrose, sorbitol, fructose, corn syrup, xylitol, glycerin, glucose, sucrose and/or other suitable agents. The solvent may contain a buffer, such as citrate, sodium or potassium phosphate and/or other such buffer known to those of skill in the art at, at about neutral pH. Subsequent sterile filtration of the solution followed by lyophilization under standard conditions known to those of skill in the art provides the desired formulation. In some embodiments, the resulting solution will be apportioned into vials for lyophilization. Each vial will contain a single dosage or multiple dosages of the Peganum harmala extract. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carriers.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration. In some embodiments, the vehicle is coconut oil although other excipients may also be used.

The Peganum harmala extract may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the Peganum harmala extract alone or in combination with other excipients can also be administered.

For nasal administration, the preparation may contain an esterified phosphonate compound dissolved or suspended in a liquid carrier, in particular, an aqueous carrier, for aerosol application. The carrier may contain solubilizing or suspending agents such as propylene glycol, surfactants, absorption enhancers such as lecithin or cyclodextrin, or preservatives.

Solutions, particularly those intended for ophthalmic use, may be formulated as 0.01%-10% isotonic solutions, pH about 5.74, with appropriate salts.

Other routes of administration, such as transdermal patches, including iontophoretic and electrophoretic devices, and rectal administration, are also contemplated herein.

Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433 and 5,860,957.

The Peganum harmala extract may be packaged as articles of manufacture containing packaging material, the Peganum harmala extract provided herein, which is effective for treatment, prevention or amelioration of one or more symptoms of the diseases or disorders, supra, within the packaging material, and a label that indicates that the Peganum harmala extract is used for the treatment, prevention or amelioration of one or more symptoms of the diseases or disorders, supra.

The articles of manufacture provided herein contain packaging materials. Packaging materials for use in packaging products are well known to those of skill in the art. See, e.g., U.S. Pat. Nos. 5,323,907, 5,052,558 and 5,033,252. Examples of packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of Peganum harmala extract provided herein are contemplated as are a variety of treatments for any disease or disorder described herein.

### Dosages

Peganum harmala extract or pharmaceutical compositions thereof, are administered or applied in a therapeutically effective amount. In human therapeutics, the physician will determine the dosage regimen that is most appropriate according to a preventive or curative treatment and according to the age, weight, stage of the disease and other factors specific to the subject to be treated. The amount of active ingredient in the formulations provided herein, which will be effective in the treatment of lichen sclerosus will vary with the nature and severity of the disease or condition, and the route by which the active ingredient is administered. The frequency and dosage will also vary according to factors specific for each subject depending on the specific therapy (e.g., therapeutic or prophylactic agents) administered, the route of administration, as well as age, body, weight, response, and the past medical history of the subject.

Exemplary doses of a formulation include milligram or microgram amounts of the Peganum harmala extract per kilogram of subject (e.g., from about 1 microgram per kilogram to about 50 milligrams per kilogram, from about 10 micrograms per kilogram to about 30 milligrams per kilogram, from about 100 micrograms per kilogram to about 10 milligrams per kilogram, or from about 100 micrograms per kilogram to about 5 milligrams per kilogram).

In some embodiments, a therapeutically effective dosage should produce a serum concentration of active ingredient of from about 0.001 ng/ml to about 50200 μg/ml. The compositions, in other embodiments, should provide a dosage of from about 0.0001 mg to about 70 mg of Peganum harmala extract per kilogram of body weight per day. Dosage unit forms are prepared to provide from about 0.01 mg, 0.1 mg or 1 mg to about 500 mg, 1000 mg or 5000 mg, and in some embodiments from about 10 mg to about 500 mg of the active ingredient or a combination of essential ingredients per dosage unit form.

The active ingredient may be administered at once or may be divided into a number of smaller doses to be administered at intervals of time. It is understood that the precise dosage and duration of treatment is a function of the disease being treated and may be determined empirically using known testing protocols or by extrapolation from in vivo or in vitro test data or subsequent clinical testing. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Initial dosages can also be estimated from in vivo data (e.g., animal models) using techniques that are well known in the art. One of ordinary skill in the art can readily optimize administration to humans based on animal data.

Ideally, a therapeutically effective dose of the Peganum harmala extract described herein will provide therapeutic benefit without causing substantial toxicity. Toxicity of the Peganum harmala extract can be determined using standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in subjects. The dosage of the Peganum harmala extract described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition (See, e.g., Fingl et al., 1975, In: The Pharmacological Basis of Therapeutics, Ch.1, p.1).

The therapy may be repeated intermittently. In certain embodiments, administration of the same formulation provided herein may be repeated and the administrations may be separated by at least 6 hours, 12 hours, 1 day, 2 days, 3 days, 5 days, 10 days, 15 days, 30 days, 45 days, 2 months, 75 days, 3 months, 6 months or indefinitely.

### Combination Therapy

The Peganum harmala extract disclosed herein may also be used in combination with one or more other active ingredients. In certain embodiments, the Peganum harmala extract may be administered in combination, or sequentially, with another therapeutic agent. Such other therapeutic agents include those known for treatment, prevention, or amelioration of one or more symptoms associated with lichen sclerosus.

It should be understood that any suitable combination of the Peganum harmala extract and compositions provided herein with one or more of the above therapeutic agents and optionally one or more further pharmacologically active substances are considered to be within the scope of the present disclosure. In some embodiments, the Peganum harmala extract and compositions provided herein are administered prior to or subsequent to the one or more additional active ingredients.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims.

The following examples are provided for illustrative purposes only and are not intended to limit the scope of the invention.

### EXAMPLES

### Example 1: Preparation of Peganum Harmala Extract

Dry Peganum harmala seeds were crushed in a blender until at least about half the seeds were fragmented. A container was about half filled with the crushed seeds. Then sufficient absolute ethanol was added until the container is about 75% full. The container was allowed to stand for about a week and is then filtered and concentrated to provide a crude Peganum harmala extract containing just enough residual alcohol to keep it in liquid form.

### Example 2: Preparation of Peganum Harmala Ointment

Unrefined coconut oil was melted at 100° C. until completely liquified. Then, Peganum harmala extract was added to the liquid coconut oil until a ratio of about one part of Peganum harmala extract to about twenty to twenty four parts of coconut oil was achieved. The mixture was then allowed to cool to provide an ointment.

### Example 3: Use of Peganum Harmala Ointment to Treat Lichen Sclerosus

The ointment prepared in Example 2 was applied to the affected skin twice daily at approximately a twelve hour interval for about one week. Then, for patients with anogenital lichen sclerosus, the ointment was applied twice daily with additionally applications following defecation or urination for the next 3-4 weeks. Patients with genital lichen sclerosus performed additional applications after urination for the same time period. Then, the ointment was applied twice daily for 1-4 months or until symptoms either resolve completely or cease to improve any further. Finally, the patient applied the ointment once daily for 1-2 weeks, followed by 1-2 weeks of one application on 6 of the 7 days in a week, 1-2 weeks of one application on 5 of the 7 days in a week, then 4 of 7 days and so on, until the patient has applied treatment one time on one day of the week for about one to about two weeks. From then on, the patient may use the treatment on an as needed basis.

What is claimed is:

1. A Peganum harmala extract made by a process comprising the steps of:
crushing dry seeds of Peganum harmala;
adding an organic solvent to the crushed seeds to form a suspension;
allowing the suspension to rest until the suspension is a dark red color;
filtering the suspension; and
removing the solvent.

2. The extract of claim 1, wherein the solvent is ethanol.

3. A pharmaceutical composition comprising the extract of claim 1 and a pharmaceutically acceptable vehicle.

4. The pharmaceutical composition of claim 3, wherein the pharmaceutically acceptable vehicle is unrefined coconut oil.

5. The pharmaceutical composition of claim 3, wherein the ratio of Peganum harmala extract to the pharmaceutically effective vehicle is between about 1 to about 20 and about 1 to about 24.

6. A method of treating and/or preventing lichen sclerosus comprising administering to a patient in need thereof a therapeutically effective amount of the extract of claim 1.

7. The method of claim 6, wherein the therapeutically effective amount of the extract is administered topically.

8. The method of claim 7, wherein the extract is administered twice daily.

9. A method of treating and/or preventing lichen sclerosus comprising administering to a patient in need thereof a therapeutically effective amount of the pharmaceutical composition of claim 3.

10. The method of claim 9, wherein the therapeutically effective amount of the pharmaceutical composition is administered topically.

11. The method of claim 10, wherein the pharmaceutical composition is administered twice daily.

12. A method of treating and/or preventing pruritis comprising administering to a patient in need thereof a therapeutically effective amount of the extract of claim 1.

13. A method of treating and/or preventing dysuria comprising administering to a patient in need thereof a therapeutically effective amount of the extract of claim 1.

14. A method of treating and/or preventing pruritis comprising administering to a patient in need thereof a therapeutically effective amount of the pharmaceutical composition of claim 3.

15. A method of treating and/or preventing dysuria comprising administering to a patient in need thereof a therapeutically effective amount of the pharmaceutical composition of claim 3.
